(19) United States
(12) Patent Application Publication
(10) Pub. No.: US 2025/0041460 A1
(43) Pub. Date: Feb. 6, 2025

(54) LANTHANIDE-BASED CONTRAST AGENTS HAVING IMPROVED STABILITY AND METHODS FOR THEIR PRODUCTION AND PURIFICATION

(71) Applicant: Inventure, LLC, Southbury, CT (US)

(72) Inventors: Richard J. DESLAURIERS, Woodbury, CT (US); Michael MILBOCKER, Holliston, MA (US); Paul LOMBARDO, Stamford, CT (US)

(73) Assignee: Inventure, LLC, Southbury, CT (US)

(21) Appl. No.: 18/794,861

(22) Filed: Aug. 5, 2024

Related U.S. Application Data
(60) Provisional application No. 63/517,763, filed on Aug. 4, 2023.

(51) Int. Cl. A61K 49/10; C07F 5/00

(52) U.S. Cl. CPC A61K 49/108; C07F 5/003

(57) ABSTRACT

The present disclosure relates to pharmaceutical formulations of contrast agents used in magnetic resonance imaging (MRI) and methods of making and purifying the same. More specifically, disclosed are such pharmaceutical formulations of chelate complexes with lanthanide metal ions, such as gadolinium ions, as well as methods for obtaining and purifying the same. In particular, the pharmaceutical formulations are free or substantially free of water molecules coordinated with the lanthanide metal chelate complexes and provide pharmaceutical compositions that are more stable and release less lanthanide ion, specifically gadolinium ion, into the human body when injected as MRI contrast agents as compared to other typically used MRI contrast agents.

### CROSS-REFERENCE TO RELATED APPLICATIONS

This application claims the benefit of U.S. Provisional Patent Application No. 63/517,763 filed Aug. 4, 2023, which is incorporated by reference herein in its entirety.

### BACKGROUND OF THE DISCLOSURE

### 1. Field of the Disclosure

The present disclosure is directed to pharmaceutical formulations of contrast agents used in magnetic resonance imaging (MRI) and methods of making and purifying the same.

### 2. Description of Related Art

Contrast agents are used in diagnostic imaging to increase the contrast and facilitate imaging of structure and fluids in the human body.

Commonly used MRI contrast agents are based on paramagnetic lanthanide series metals, and particularly gadolinium. Chelated complexes of gadolinium include, for example, macrocyclic chelation compounds such as 1,4,7,10-tetraazacyclo-dodecane-N, N′, N″, N′″-tetraacetic acid (DOTA), 2,2′,2″-[10-(2-hydroxypropyl)-1,4,7,10-tetraazacyclododecane-1,4,7-triyl]triacetic acid (HPDO3A), diethylenetriamine (DTPA), 1,4,7,10-tetraazacyclododecane-1,4,7-triacetic acid (DO3A), 1,4,7-Triazacyclononane-1,4,7-triacetic acid (NOTA), 2-[1,4,7,10-tetraazacyclododecane-4,7,10-tris(t-butyl acetate)]-pentanedioic acid-it-butyl ester (DOTAGA), 3,6,9,15-tetraazabicyclo[9.3.1]pentadeca-1(15),11,13-triene-3,6,9-triacetic acid (PCTA), 10-[(2R,3S)-2-hydroxy-3-hydroxy-1-(hydroxymethyl)propyl]-1,4,7,10-tetraazacyclododecane-1,4,7-triacetic acid (BTDO3A), and 2-[6,9-bis({1-carboxylato-3-[(2,3-dihydroxypropyl)carbamoyl]propyl})-3,6,9,15-tetraazabicyclo[9.3.1]pentadeca-1(14),11(15),12-trien-3-yl]-4-[(2,3-dihydroxypropyl)carbamoyl]butanoic acid.

Lanthanide chelate complexes occur predominately in the metal ligand chelated form when first introduced into the human body. These lanthanide chelate complexes are in chemical equilibrium with their unchelated free lanthanide metal ionic forms. However, over time these lanthanide chelate complexes release unchelated lanthanide ions into the body. For example, lanthanide chelate complexes based on gadolinium release gadolinium ion.

The environment of the body has a destabilizing effect on the lanthanide metal chelate complexes. Free lanthanide ions in the body can be highly toxic, even in low amounts. Moreover, risks associated with free lanthanide ions in the body can be compounded by the often necessary repeated administration of contrast agents.

There have been prior approaches for improving the tolerability of gadolinium chelate complexes.

For example, one approach has been adding an excess of a chelating compound to a lanthanide based contrast agent to reduce the amount of free gadolinium ion in the drug product prior to injection. However, this is not an ideal solution. For one reason, the chelating compound itself is toxic; thus, adding excess chelate is not desirable. Further, while excess chelate may achieve a reduced gadolinium ion formation upon manufacturing and storage prior to injection, the excess chelate does not correlate to a reduced gadolinium ion release in the body. Ultimately, the excess chelating agent does not significantly change the equilibrium between free gadolinium ions and its chelated form and therefore, does not solve the problem of the release of free gadolinium ion. Further, a small number of gadolinium chelates present with the excess chelate are metastable. These metastable chelate complexes readily release free gadolinium when they are introduced or injected into the body. While there may be a perceived safety enhancement with respect to drug product testing and characterization, such safety enhancement is not, in fact, realized in practice when the chelates are injected into the human body. This is because the reduction of gadolinium ions in the drug product does not correlate with a reduction in the release of free gadolinium ions in the body. The drug product environment prior to administration, for example conditions in a bottle, are very different than conditions in the human body.

In other approaches, a chelate compound such as a ligand (L) can be added in excess with respect to a macrocyclic chelate compound in the form of an additive having the formula X [X′, L], where X and X′ are metal ions and L is an excess of the chelate compound. In this example, the metal ions are competitive metal ions, for example calcium, sodium, zinc, or magnesium. These additives are intended to capture free lanthanides by reacting with the metal ions X and X′ to form complexes with free gadolinium ions present in the formulation. While this approach reduces the toxicity of the free excess chelate, it shifts the gadolinium ion complex equilibrium toward releasing more gadolinium ions. While the additional release of free gadolinium ions may be taken up by the excess X [X′, L], the presence of more metastable chelates is the result due to the destabilizing effect of competitive metal ions that are introduced. In fact, this approach may generate many more metastable complexes in the original formulation than the number of free gadolinium ions present.

The commonly available contrast agents are not presently tested for the presence and quantity of metastable complexes.

Free chelate may form undesirable complexes when introduced into the body. In particular, according to the LD50 value, macrocyclic chelate compounds such as HPDO3A, DO3A, and DOTA in the form of X [X′, L] are at least about 10 times more toxic than the free macrocycles.

The safety of gadolinium contrast agents also depends on the purity of the gadolinium used. Gadolinium found in nature is associated with other heavy metals that are toxic and provide little to no benefit for diagnostic imaging.

MRI contrast agents that are based on gadolinium commonly also contain traces of Scandium (Sc), Yttrium (Y), Lanthanum (La), Cerium (Ce), Praseodymium (Pr), Neodymium (Nd), Europium (Eu), Terbium (Tb), Thulium (Tm), Dysprosium (Dy), Holmium (Ho), and Erbium (Er). In one analysis, Tb, Tm, Eu, and La were, on average, found in the highest amounts, 0.42 mg/L, 0.17 mg/L, 0.17 mg/L, and 0.16 mg/L, respectively.

Gadolinium based contrast agents (GCA) incorporating linear ligand chelation are fundamentally different from GCAs incorporating macrocyclic ligands. Macrocyclic GCAs can be synthesized by pathways characterized by the formation of a sequence of metastable complexes before obtaining the final stable complex.

Macrocyclic gadolinium-based contrast agents can be synthesized by pathways characterized by the formation of a sequence of metastable (i.e., intermediate) complexes before obtaining the final stable complex. This allows for the production of aqueous or dry/solid GCA with near zero metastable species content.

U.S. Patent Publication 2011/0129425 discloses a process for preparing a liquid pharmaceutical formulation said to have reduced free lanthanide. This formulation is prepared with a lanthanide and a molar excess of free macrocyclic chelator between 0.002 and 0.4%. The macrocyclic chelator is chosen from DOTA, DOTAGA, DO3A, BTDO3A, HPDO3A, and PCTA.

Accordingly, it has been determined by the present disclosure that there is a continuing need for compositions and methods for preparing compositions of lanthanide metal complexes with macrocyclic chelators which do not contain free lanthanide metal ion and do not contain metastable lanthanide metal complexes which release free lanthanide metal ion when introduced into a mammalian body and that overcome, alleviate, and/or mitigate one or more of the aforementioned and/or other deleterious effects that were the target of prior attempts.

### SUMMARY

In general, the present disclosure is directed to pharmaceutical formulations of contrast agents used in magnetic resonance imaging (MRI) and methods of making and purifying the same.

More specifically, the present disclosure is directed to such pharmaceutical formulations of chelate complexes with lanthanide metal ions, preferably gadolinium ions, as well as methods for obtaining and purifying the same.

In particular, the pharmaceutical formulations are free or substantially free of excess water molecules coordinated with the lanthanide metal chelate complexes.

The present disclosure provides methods for making and for purifying compositions, and the resulting compositions, of lanthanide metal complexes that are particularly well-suited for providing MRI contrast agents containing such formulations.

In an embodiment according to the present disclosure, the methods for making the lanthanide metal complexes comprise making a lanthanide series ion DOTA contrast agent including the steps of:
(a) adding DOTA to purified water, for example, distilled or deionized water, in a vessel to form a solution or suspension;
(b) stirring this mixture and raising the temperature of the mixture to a temperature of 92° C. to 98° C., preferably about 95° C.;
(c) adding a lanthanide series oxide incrementally to the mixture, wherein each incremental addition is made when the mixture from the previous addition turns substantially or completely clear; and
(d) repeating (c) until a substantially equimolar amount of lanthanide series oxide is added as compared to the DOTA.

The method may also include:
(e) reacting the lanthanide oxide DOTA solution at a temperature of 92° C. to 98° C., preferably at about 95° C. and for at least 24 hours, preferably at least 36 hours, for example 24 hours to 40 hours, to obtain a lanthanide oxide DOTA complex. At this point, the TSA structure of the lanthanide ion DOTA complex is present and can be separated and collected.

Thereafter, the lanthanide oxide DOTA complex may be purified and meglumine can be added to the purified product until the pH is about 7.2 to about 7.4.

In some embodiments, the solution of (e) is cooled to a temperature of 35° C. to 45° C., preferably about 40° C. before the meglumine is added. In some embodiments, the cooling is performed at a rate that is faster than ambient cooling, such as with a heat exchanger. In some embodiments, the lanthanide oxide is gadolinium oxide. At this point, the TSA′ locked structure of the lanthanide ion DOTA complex is present and can be separated and collected. As used herein, the term TSA′ locked structure refers to the lowest potential energy state of the system.

In an embodiment according to the present disclosure, the methods for purifying lanthanide metal ligand complexes comprise: (a) dissolving the lanthanide metal ligand complex in a solvent to obtain a lanthanide metal ligand solution, (b) placing the lanthanide metal ligand solution in a first side of a chamber partitioned into two sides by a semipermeable membrane, wherein the second side contains a solvent, (c) applying a static magnetic field sufficient to cause the lanthanide metal ligand complex to aggregate, (d) lowering the temperature of the solution to below the Curie temperature of the lanthanide metal-ligand complex, and (e) removing the purified lanthanide metal ligand solution from the first side. In some embodiments, the first and second solution contain an electrolyte, and an electric field is applied. In some embodiments, the semipermeable membrane has a porosity in the range 1 to 50 kDa, more preferably 0.5 to 1 kDa. The second side may also contain an ionic gel suitable for sequestering metal ions, and free metal ions are removed from the first side and captured by the ionic gel in the second side. In some embodiments, more than 99% of free metal ion and non-ferromagnetic metal ligand complex is removed from the first side to the second side. In some embodiments, the purified lanthanide metal ligand obtained from the first side is stabilized by the addition of meglumine.

In an embodiment, the present disclosure provides a method of obtaining a gadolinium ligand complex having a TSA structure comprising (a) dissolving the gadolinium ligand complex in a solvent to obtain a gadolinium ligand solution, (b) placing the gadolinium ligand solution in a first side of a chamber partitioned into two sides by a semipermeable membrane, wherein the second side contains a solvent, (c) adding a challenging liquid chelate to the first side, (d) applying an electric field that drives free metal ions across the semipermeable membrane to the second side, (e) placing a solid ionic gel on the second side, (f) immobilizing metal ions on the ionic gel, and (g) removing the TSA structured gadolinium ligand solution from the first side.

In an embodiment, the present disclosure provides a lanthanide series ion DOTA contrast agent comprising (a) a plurality of lanthanide ions; (b) a plurality of DOTA chelate molecules, wherein the plurality of lanthanide ions and the plurality of DOTA chelate molecules form lanthanide ion DOTA complexes, wherein the lanthanide ion DOTA complexes are in a TSA structure. In some embodiments, the lanthanide series ion is Gd³⁺. In some embodiments, greater than 95% of the complexes of gadolinium and DOTA possessing a TSA structure referred to as aquaoctakis(1,4,7,10-tetraazacyclododecane-1,4,7,10-tetraacetic acid)gadolinium(III). In some embodiments, the complexes further comprise meglumine, wherein the meglumine locks the structure of the lanthanide ion DOTA complexes in a TSA′ structure and wherein none of the coordination sites of the lanthanide ion are coordinated to a water molecule. In some embodiments, the lanthanide series ion is Gd³⁺. In some embodiments, greater than 95% of the complexes of gadolinium and DOTA possess a TSA′ structure, wherein the TSA′ structure has no waters in the coordination sphere. As noted above, this TSA′ structure is also referred to herein as TSA′ Gd Product.

In an embodiment, the present disclosure provides a lanthanide series ion DOTA contrast agent comprising: (a) a plurality of lanthanide ions, (b) a plurality of DOTA chelate molecules, wherein the plurality of lanthanide ions and the plurality of DOTA chelate molecules form lanthanide ion DOTA complexes; and meglumine, wherein the lanthanide ion DOTA complexes release relatively few gadolinium ions as compared to Dotarem® (Guerbet LLC) and Clariscan® (GE HealthCare). In testing, Dotarem® and Clariscan® released 5000-10000 ppm (mol wt. %) more gadolinium ions than the lanthanide ion complex disclosed here when subjected to a competitive ion scavenging agent such as Purolite® C150 resin for a period of about 2 hours. Purolite® C150 is Polystyrenic Macroporous, Strong Acid Cation Resin, Sodium form, resin with a macroporous polystyrene cross-linked with divinylbenzene polymer structure. In some embodiments, the lanthanide series ion is Gd³⁺.

In an embodiment, the present disclosure provides a lanthanide series ion DOTA complex with locked TSA′ structure and having no coordinated water with a stability greater than the stability of a lanthanide series ion DOTA complex containing at least one water molecule. In some embodiments, the lanthanide series ion is Gd³⁺. In some embodiments, the gadolinium contrast agent or TSA′ Gd Product has a log stability constant (conditional at pH 7.4) greater than 19.3. Additionally, the gadolinium contrast agent has a log stability constant (thermodynamic at standard temperature and pressure) of greater than 25.6.

In coordination chemistry, a stability constant is an equilibrium constant for the formation of a complex in solution. It is a measure of the strength of the interaction between the reagents that come together to form the complex.

As discussed herein, the pharmaceutical formulations of contrast agents used in magnetic resonance imaging (MRI) and methods of making and purifying the same are described to comprise meglumine. Meglumine is also referred to as N-methylglucamine. Meglumine has a molecular formula of C₇H₁₇NO₅. Meglumine has a pKb of about 0.526.

Meglumine is a secondary alkanolamine. Without wishing to be bound by theory, it is believed that other amines with a suitable pKb and similar molecular size including carbohydrate amines are contemplated as an alternative to meglumine.

In other embodiments, the present disclosure relates to methods or processes for making lanthanide ion dodecane tetraacetic acid (DOTA) complex contrast agents and additional to purification methods or processes as described herein.

### BRIEF DESCRIPTION OF THE DRAWINGS

The accompanying drawings illustrate aspects of the present disclosure, and together with the general description given above and the detailed description given below, explain the principles of the present disclosure. As shown throughout the drawings, reference numerals designate like or corresponding parts.

FIG. 1 shows a comparison between the SA complex structure and the TSA complex structure according to the present disclosure.

FIG. 2A shows the atomic associations in a gadolinium DOTA complex which has a water coordinated with the Gd³⁺ and a TSA structure (left side) and free meglumine (right side) according to the present disclosure. FIG. 2B shows a gadolinium DOTA meglumine complex in which meglumine has replaced the water bound to the Gd³⁺ and has a TSA′ structure according to the present disclosure.

FIG. 3 shows the relative decomposition of two marketed MRI imaging products Dotarem® and Clariscan® normalized to the TSA′ Gd Product MRI imaging agent produced by the methods described in the present disclosure.

FIG. 4 shows the relative decomposition of commercial MRI imaging products Dotarem® and Clariscan® as compared to the TSA′ Gd Product according to the present disclosure.

FIG. 5 shows the data of FIG. 4 with a normalized curve.

FIG. 6 shows a schematic of a membrane purification system according to the present disclosure.

FIG. 7 shows Dotarem® without a resin treatment according to the disclosure.

FIG. 8 shows Dotarem® with a resin treatment according to the disclosure.

FIG. 9 shows a Gd Product prior to resin treatment according to the disclosure.

FIG. 10 shows a Gd Product after resin treatment according to the disclosure.

### DETAILED DESCRIPTION OF THE DISCLOSURE

Lanthanide DOTA complexes can occur in a variety of structures with different kinetic stabilities, and that these structures can vary as a function of the complexation time. The less stable structures are formed first. The DOTA ligand is bound octadentately to the Ln³⁺ ion through the four N-atoms and an O-atom of each of the four carboxylate groups. The binding of the Ln³⁺ ion by the N-atoms fixes the four ethylene groups of the macrocycle in two enantiomeric helicity conformations having all of these groups either in a delta or a lambda gauche helicity orientation. The four acetate arms can be arranged with opposite helicities. The combination of all the metal binding units in one ligand defines an Ln³⁺ coordination polyhedron formed by two parallel faces defined by the four ring nitrogen atoms (N4 plane) and four acetate arm oxygen donor atoms (O4 plane) of the ligand with a twist angle (x) and a distance (d) between those planes.

The O-Ln-O angle between two transannular oxygen atoms in the O4 plane is known as the opening angle (w), which correlates to the dissociation energy barrier of the lanthanide ion from DOTA. The twist angle and distance between planes comprise the stereochemical elements that combine to form two diastereoisomeric pairs of enantiomers, with the ring and the arms having opposite helicities, leading to a square antiprismatic (SA) coordination geometry (x positive), and the arms with the same ring an acetate helicity, giving a twisted square antiprismatic (TSA) structure (x negative). The coordination polyhedron may be extended to have a single water O-atom capping the O4-plane. Without such a water molecule, the respective complex geometries are called SA′ and TSA′. These prime structures become important when meglumine is added to a solution containing SA and TSA structures.

Lanthanide DOTA complexes have a nine coordinate structure with water molecules in the first coordination sphere of the Ln³⁺ ion and SA geometry (x˜39°).

However, the structures obtained for the complexes when meglumine is present in solution are different. In this higher pH environment, the lanthanide complex has a polymeric structure with a TSA′ geometry (x=−22°), with no lanthanide bound water but instead a bridging carboxylate O-atom of a neighboring complex molecule. The average opening angle determines whether or not a water molecule is coordinated. As the opening angle increases there is more space available to coordinate water. The complex is less stable as more water is coordinated. For example, the TSA configuration has opening angle of 144°, whereas the SA configuration has opening angle 148° (see, FIG. 1). Thus, even the water molecule in the first coordination sphere is destabilizing, less so in the TSA configuration as compared to the SA configuration. The addition of meglumine enables the TSA′ configuration, which is optimally stable for the gadolinium complex with DOTA.

The structures of Gd³⁺ complexes initially correspond to the SA isomer. If a free gadolinium measurement is made at this stage of the complexation, the free gadolinium levels will be measured to be low, suggesting the complexation is complete. While this is true, these SA isomers correspond to complexes with water in the coordination sphere. Typically, there can be as many as six water molecules in the coordination sphere of the complex. As the complex solution is further heated, the structure of this isomer changes in stepwise variation of the torsion angle Ln-N—C—COO until the fit of the metal ion in the ligand is constrained. For a preferred fit, the arrangement of the acetate arms corresponds to a TSA structure with an orientation of the acetate arms that is inverted with respect to the main isomer, thus with a negative and smaller angle of rotation between the N4- and O4-planes.

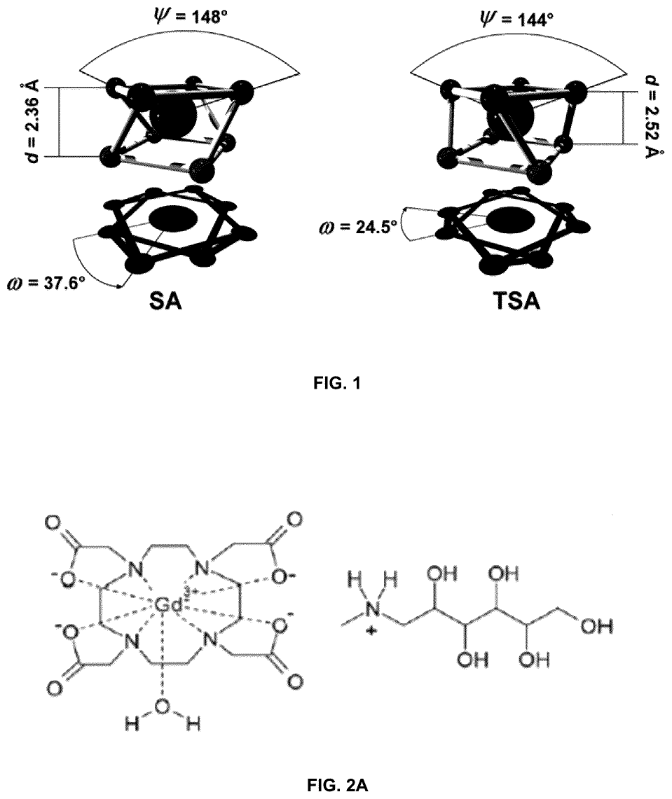

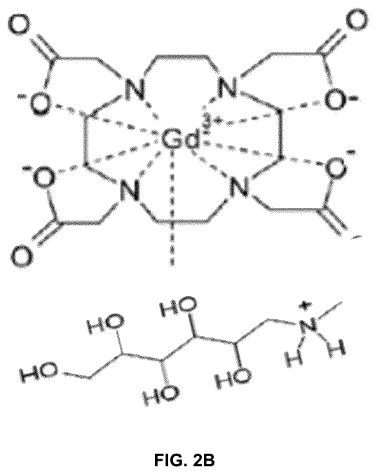

After the TSA structure is achieved it can be locked in by the addition of a meglumine, transitioning the TSA structure to a TSA′ structure. The TSA′ structure is referred to at times herein and in the Figures as “TSA′ Gd Product”. FIG. 2 shows the TSA structure prior to the addition of meglumine where the Gd³⁺ has a water molecule bound to it. The TSA structure is converted to the TSA′ structure after the addition of meglumine that locks the complex in the TSA′ structure having no water molecules coordinated to the complex.

The gradual decrease of the ionic radius across the lanthanide series is accompanied by an increasing steric strain in the complexes and charge density of the cations. These counteracting effects dominate the coordination chemistry of the lanthanides. The unrecognized outcome of these counteracting effects is a trend to gradual changes in the coordination polyhedra during complexation. This observation explains the bias toward the earlier SA complex structure since the United States Pharmacopeia-accepted diagnostic methods do not discriminate between SA and TSA structures. While these structures result in similar drug product free gadolinium ion content assays, free gadolinium is rapidly released in a ligand destabilizing environment, as is found in living systems. In the body, the bound ligand conformations, complex stabilities, and dynamics, are often accompanied by changes in coordination numbers.

Chelation of a lanthanide metal with a macrocyclic ligand frequently results in a spectrum of complexes, wherein some of the coordination sites are occupied by water. When a maximum number of coordination sites on the lanthanide metal ion are occupied by groups on the ligand, the resulting complex is defined as “fully complexed”. For example, in the case of forming complexes between the lanthanide metal gadolinium and the ligand DOTA there are nine coordination sites on the gadolinium ion, eight of which are occupied by groups on the ligand DOTA. The ninth site is occupied by an external water molecule.

Gadolinium with a fully coordinated DOTA ligand and one neutral water ligand is referred to as aquaoctakis(1,4,7,10-tetraazacyclododecane-1,4,7,10-tetraacetic acid)gadolinium(II). Frequently, this complex is further reacted with meglumine to obtain aquaoctakis(1,4,7,10-tetraazacyclododecane-1,4,7,10-tetraacetic acid)gadolinium(II) meglumine complex.

Here, the present disclosure distinguishes aquaoctakis(1,4,7,10-tetraazacyclododecane-1,4,7,10-tetraacetic acid)gadolinium(III) from the more common molecules containing two or more neutral waters, ligand bonded to gadolinium. These less stable complexes must be actively removed from a reaction solution of water, gadolinium oxide, and DOTA.

Also, under appropriate conditions described herein, aquaoctakis(1,4,7,10-tetraazacyclododecane-1,4,7,10-tetraacetic acid)gadolinium(III) can be free of coordinated water under conditions where meglumine replaces the one neutral water of the named aquaoctakis(1,4,7,10-tetraazacyclododecane-1,4,7,10-tetraacetic acid)gadolinium(III).

Neutral water ligands associated with the complex makes it easier to dissociate gadolinium ions from the DOTA, thereby decreasing the stability of the complex. These less stable complexes can be referred to also as metastable. In the bottle, these less stable complexes remain in a complexed form, resulting in the low free gadolinium ion content reported in the literature and listed on the bottle containing these complexes.

However, in the body, where many competing metal ions are present, these metastable complexes readily dissociate, causing the potential free gadolinium delivered to the body to be several orders of magnitude higher than measured in the bottle. In addition, hydroxyl groups, other than those on DOTA, should not be present during the complexing process. For example, as disclosed herein, adding meglumine early in the complexation process would give the impression that the complexation process is complete before all the complexes are in the preferred aquaoctakis(1,4,7,10-tetraazacyclododecane-1,4,7,10-tetraacetic acid)gadolinium(Ill) form. On the other hand, adding meglumine later in the complexation process, and under conditions described herein, ensures that most or all of the neutral water molecules are removed. A further complication in making these complexes is that the lanthanide metals are very difficult to separate. Consequently, gadolinium oxide often is associated with oxides of other lanthanide metals.

The size of the ions of rare-earth elements steadily decreases with increasing atomic number from lanthanum (atomic number 57) through lutetium (atomic number 71). The gradual decrease of water exchange rates and increase of kinetic stability constant of heavy lanthanides going from Gd³⁺ to La³⁺, as the ion size increases, is an indication that the larger the ion size, the greater the steric fit in the coordination cage of a polydentate ligand. This implies there is a reciprocal relationship between relaxation rate (MRI signal intensity) and complex stability. Kinetic stability, in the context discussed here, is a measure of how difficult it is to disassociate a complex. A similar measure is the thermodynamic stability constant, which is a measure of the energy difference before and after complex dissociation.

FIG. 1 shows schematically the differences between the SA complex structure and TSA structure, as discussed in detail above. In general, the SA structure is that present when two or more water molecules are coordinated to the gadolinium chelate complex. The more coordinated water, the less stable the complex. The TSA structure has only one coordinated water molecule and, as can be seen in FIG. 1, the intramolecular angles in the TSA structure are smaller than those of the SA structure. In addition, the intramolecular distances are also smaller in the TSA structure as compared to the SA structure. These differences contribute to the increased stability of the TSA structure as compared to the SA structure.

FIGS. 2A and 2B show the difference between the TSA structure and the TSA′ structure. In FIG. 2A, the structure of Gd³⁺ DOTA meglumine complex having one coordinated water molecule is shown. In FIG. 2A, the complex is not chelated with meglumine. In FIG. 2B, the meglumine has replaced the coordinated water shown in FIG. 2A, resulting in the Gd³⁺ DOTA meglumine complex free of coordinated water. Both of these complexes can be made and purified according to the processes described herein.

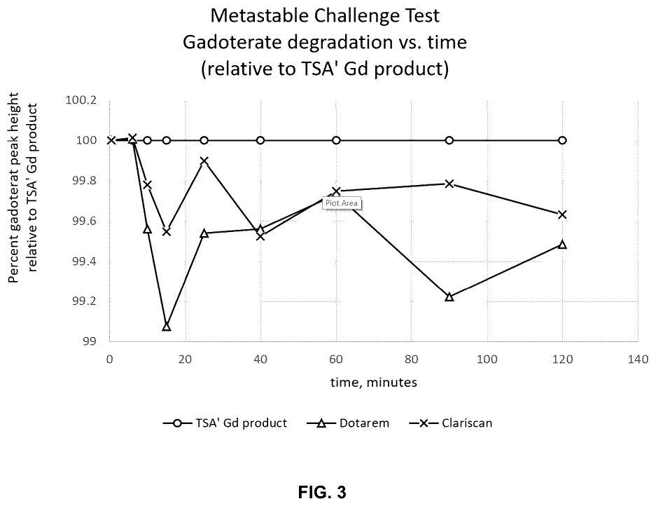

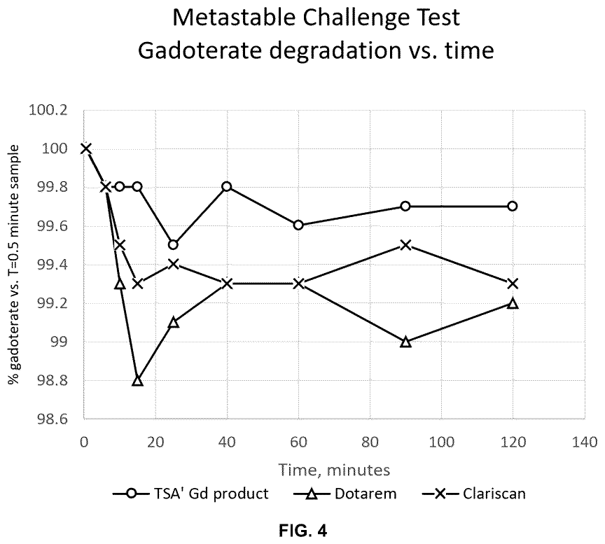

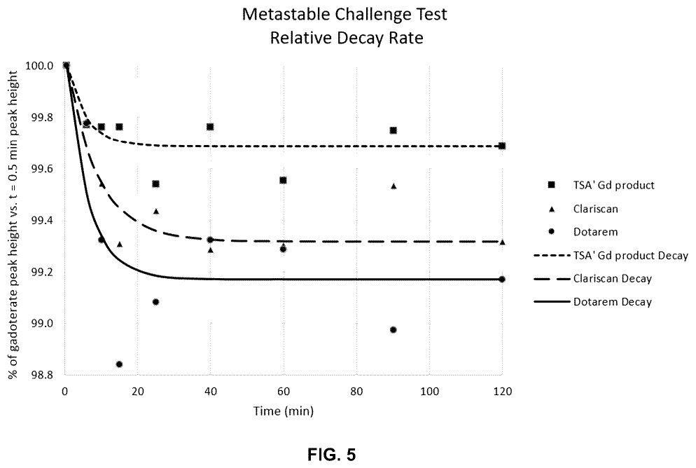

FIGS. 3 and 4 show the relative and absolute stability of Applicant's disclosed TSA′ Gd Product as compared to two commercially available and frequently used MRI contrast agents Dotarem® and Clariscan®. The data shown in FIGS. 3 and 4 were obtained as follows. Exposing the product to a cation retaining resin, any free gadolinium ions in the sample are retained on the resin. Since the free gadolinium ions are continually removed, the equilibrium that would normally slow the decomposition of gadolinium DOTA metastable compounds is shifted toward decomposition. The gadolinium DOTA complexes that have the more stable TSA′ structure are slow to decompose even with the cation attracting resin present. Gadolinium DOTA complex treated with xylenol orange has a characteristic peak at 274 nm in the ultraviolet spectrum. The amount of metastable gadolinium DOTA complex in the mixture can be inferred by measuring the decrease in this peak height with an ultraviolet spectrometer. FIG. 3 shows the relative contrast agent stability as measured over time where the percentage gadolinium DOTA complex peak height is relative to the measurement of the gadolinium DOTA complex synthesized and described in this document. FIGS. 4 and 5 shows the comparative absolute relative stability for each MRI contrast agent. As can be seen, TSA′ Gd Product is more stable as compared to Dotarem® and Clariscan®.

The test procedure used to obtain the foregoing results was as follows:
1. Expose the resin to water for 24 hours.
2. Measure the 274 nm peak height of the sample just prior to exposure to resin. This is the initial time point.
3. Combine one equivalent of resin with one equivalent of the sample and agitate.
4. Sample the liquid portion of the mixture and immediately measure the 274 nm peak height with aa ultraviolet spectrometer at 30 seconds, 6 minutes, 9 minutes, 12 minutes, 15 minutes, 30 minutes, 60 minutes, and 120 minutes.
5. Record the 274 nm peak height corrected for the water blank sample.

It has been found, surprisingly, that increasing the size of the ligand cage improves the stability of the lanthanide ligand complex as the charge density increases and the ionic size decreases. Without wishing to be bound by theory, a possible reason for this is that the opening angle decreases as the cage volume increases, resulting in steric hindrance to ion dissociation from the ligand. Of all the lanthanide metals, gadolinium has the highest water exchange rate.

The Examples set forth below are illustrative of the present disclosure and are not intended to limit, in any way, the scope of the present disclosure.

### EXAMPLES

### Example 1. Removal of Metal Ion, Gadolinium Ligand Metastables, and Non-Ferromagnetic Metal Ligand Complexes from a Solution of Gadolinium Ligand Complexes Using a Semipermeable Membrane and Static Magnetic Field

The Curie temperature of gadolinium is 19° C., below which gadolinium transitions from a paramagnetic metal to a ferromagnetic metal. When a static magnetic field is applied to a solution of gadolinium ligand complex, the gadolinium ions coordinated in the macrocyclic ligand align their magnetic dipoles to the applied magnetic field. The applied magnetic field causes the gadolinium ligand complex to become attractive, and individual gadolinium ligand complexes will associate into clumps or aggregates measuring orders of magnitude larger than a single gadolinium ligand complex. This clumping or aggregating is non-trivial when used in conjunction with size exclusion membranes. The exclusion probability increases rapidly with the increasing size of the particle to be excluded from diffusion through the membrane.

Free gadolinium ions easily pass through a size exclusion membrane. Commercially available size exclusion membranes can exclude molecules in the range of 0.1 to 1,000 kDa, but they are far more efficient when the species to be excluded is above approximately 5 kDa. Gadolinium-ligand complexes are typically below 1 kDa, for example, the molecular weight of Dotarem® is 754 Da. Commercially available gadolinium oxide used in making gadolinium ligand chelates contains other transition metals that are known to be toxic and provide little therapeutic value. They also typically form fewer stable complexes with macrocyclic ligands since ionic size is very important in determining the kinetic stability of the metal-ligand complex. All of these metals contaminating gadolinium have Curie temperature far below the freezing temperature of water, and consequently will be paramagnetic in the temperature range of 0 to 19° C. in which an aqueous solution of gadolinium ligand is ferromagnetic. In this temperature range the gadolinium ligand complex will clump or aggregate when a static magnetic field is applied, and the other metal ligand complexes will not. Hence, being able to increase the pore size of the size exclusion membrane to allow these contaminating metal ligand complexes through the membrane, while excluding the clumped or aggregated gadolinium ligand complexes, is of significant importance.

When an aqueous solution of gadolinium ligand complex containing other metal ligand complexes is placed on one side of a size exclusion membrane and a magnetic field is applied, the gadolinium ligand complex clumps or aggregates and is segregated from the other side of the membrane. If an ionic gel is placed on the other side of the membrane, then complexes passing through the membrane will either be directly captured by the gel or the gel will cause the metal ion to disassociate from the ligand and attach to the gel. In either case, when a contaminating metal ligand complex passes through the semipermeable membrane by diffusion, it is prevented from diffusing back across the membrane by being localized on a solid phase ionic gel.

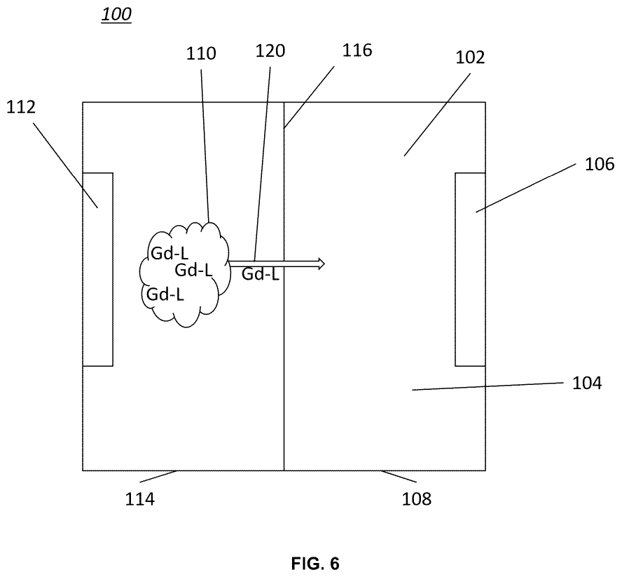

FIG. 6 illustrates a membrane purification system 100, comprising an optional electrolytic cell 102 containing an electrolyte 104 on the electrode 106 that is an anode side 108, for example meglumine, and a complex 110 of gadolinium ligand in aqueous solution, for example gadolinium DOTA, on the electrode 112 that is a cathode side 114. A semipermeable membrane 116 separates sides 114 and 108. The porosity of the semipermeable membrane 116 is chosen to allow non-clumped or aggregated free ligand 120 across semipermeable membrane 116, but to exclude transport of the clumped or aggregated complex 110. To complete the setup, the solution is cooled in the range of 0 to 19° C., and a strong magnetic field is applied. The strength of the field is typically one tesla or greater. Optionally, an electric field gradient can also be employed to draw ions across and through the membrane, for example, transition and rare earth metal ions, including gadolinium. The electric field can significantly increase the probability that the metal ions cross the membrane before recombining with the ligand.

It will be appreciated that the above example is useful in removing free metal ions, contaminating metal ligand complexes, and gadolinium ligand complexes which are metastable. The metastable gadolinium ligand complexes have gadolinium ions that spend more time in the free ionic state, which in conjunction with an applied electric field, ensures that the metastable complex will dissociate, and their ions pass across the semipermeable membrane before they can re-complex.

The semipermeable membrane purification process described above can also be performed without electrolysis. If the complex 110 is metastable, the complex 110 will eventually decay into free lanthanide ion and free ligand. The time for a metastable complex to disassociate is much shorter than the time for a fully coordinated complex to disassociate. If there is a lanthanide sequestration means, for example a solid state cation resin, cationic resin 124, the free lanthanide can be removed from the solution before it complexes with any free ligand 120 present. The semipermeable membrane purification process may have the cationic resin 124 located on the anode side 108.

Typically, the lanthanide ion 126 is small enough to pass through semipermeable membrane 116. The advantage in this arrangement is that the cationic resin 124 need not be in a solid state if the semipermeable membrane 116 excludes transport of the cationic scavenger across cationic resin 124 to cationic side 114.

Time, heat, and pH can be used to challenge the complexes 110, encouraging them to disassociate. Preferably, the challenge does not cause stable complexes to disassociate. Electrolysis provides a quantifiable, and more rapid way to disassociate metastables of complexes 110 and remove them differentially from a solution of fully coordinate complexes. When electrodes 106 and 112 are charged, metastables of complex 110 are disassociated 128 on electrode 112. Lanthanide ion 126 is then deposited 130 on electrode 112. All these semipermeable membrane purification procedures will remove heavy and transitional metal contaminants. The electrolysis method is preferred since metal contaminants may be present as a metastable complex comprised of the metal contaminant with the ligand 120.

Toxic metals removed by the present purification procedures include Sc, Y, La, Ce, Pr, Nd, Eu, Tb, Tm, Dy, Ho, and Eu. All of these metals have been found in commercially available, high purity gadolinium oxides. Terbium, thulium, europium and lanthanum have been found in the highest amounts, which were 0.42, 0.17, 0.17, and 0.16 mg/L, respectively.

Generally, a membrane electrolysis cell comprises cathode and anode, which can be divided by a cation exchange membrane which prohibits negatively charged ion transport between the electrolytes. In this cell, the electrolyte solution surrounding the cathode is known as catholyte and the electrolyte near the anode is called anolyte. Catholyte electrolyte contains metal and chelating ligands, whereas anolyte electrolyte consists of sodium chloride. During membrane electrolysis, Na+ ions from the anolyte solution diffuse and carry the current through the cation-exchange membrane into the catholyte solution to complete the electrical circuit of the process.

### Example 2. Synthesis and Purification of TSA Aquaoctakis(1,4,7,10-Tetraazacyclododecane-1,4,7,10-Tetraacetic Acid)Gadolinium(III)

One mole of DOTA is added to 1 L of distilled water in a vessel with a stirring mechanism. The reaction temperature raised to 95° C. When that temperature is reached, gadolinium oxide is added to the reaction volume in incrementally. Gadolinium oxide additions are made when the reaction solution turns clear, or the amount of free DOTA reaches 0.005 vol % to 0.05 vol % on a final product basis, and free gadolinium ion is preferably less than 10 ppm mol-wt, more preferably less than 1.5 ppm mol-wt, and most preferably less than 1.0 ppm mol-wt on a final product basis. The reaction mixture is reacted at 95° C. for at least 24 hours, preferably 32 hours. This extended heating is a necessary step in converting the SA form of the gadolinium ligand complex to the TSA form.

This reaction product can be purified using the setup of Example 1. It is important to perform this purification before meglumine addition, since adding the meglumine will tend to stabilize the non-gadolinium chelates intended to be removed by de-chelation and passage of the resulting metal ions across the semipermeable membrane. This can be done by using an electric field, a magnetic field, dielectrophoresis or by relying on diffusion alone. The purified solution is collected from the first chamber and optionally further purified using the method of Example 3. The purified gadolinium ligand solution cooled to about 40° C. and is then stabilized by the addition of meglumine until the pH reaches the intended pH target, usually in the range of 7.2 to 7.4. The meglumine addition locks the gadolinium within the macrocyclic cage of the DOTA by transforming the TSA structure into the TSA′ structure. The resulting drug product is both stable in the bottle, but more importantly is stable when injected into a living body, where trans-metalation and competitive chelation can occur.

### Example 3. Magnetic Field Gradient Contaminating Metal Removal

Gadolinium is the only lanthanide series metal with a Curie temperature above the freezing point of water. Gadolinium has a ferromagnetic Curie point of 20° C. (68° F.). In this example, the Curie point, the transition temperature below which a metal transitions from a weakly magnetic paramagnetic state to a strongly magnetic ferromagnetic state is used. Transition from a paramagnetic state into a ferromagnetic state causes gadolinium ions to clump or aggregate together in the presence of a magnetic field and be attracted along a magnetic field gradient. In Example 1, this clumping or aggregating effect is employed to ensure the gadolinium complex clumps or aggregates into large size aggregations to prevent gadolinium ligand complex from passing through a semipermeable membrane separating the solution to be purified from a chamber containing an ionic gel capable of localizing gadolinium ion.

In the present example, a rare earth magnet possessing a field greater than 1 tesla has a cylindrical shape having a central hole situated parallel with the magnetic field. The magnetic field inside the central hole is more intense than the magnetic field outside the central hole. More generally, the intensity of the magnetic field steadily drops as a function of distance from the central hole. The central hole can be extended using a hollow cylinder connected to the central hole. Such an arrangement better defines a separation volume with a unidirectional magnetic field gradient.

In a commercially prepared gadolinium ligand aqueous solution, typically there are contaminating metal-ligand complexes. These contaminating metals are typically associated with gadolinium sources and are not removed from gadolinium raw materials to the degree desired. In solution, commercially prepared gadolinium ligand aqueous solution is purified using the present example by placing the magnet assembly in the solution when the solution temperature is below the Curie temperature. The gadolinium ligand complex becomes ferromagnetic, whereas the other metal ligand complexes do not. Accordingly, the gadolinium ligand complex aggregates, resulting in an attraction toward the magnet larger than if the gadolinium ligand complex were not aggregated. As a result of thermal diffusion, over time the concentration of the gadolinium ligand complex increases as a function of closeness to the magnet, and the concentration of metal-ligand complex decreases. The decrease is due to osmotic processes, where the total concentration of complexes remains constant throughout the solution. Consequently, as the gadolinium ligand concentration increases the metal-ligand concentration must decrease. After sufficient time, the gadolinium ligand complex can be selectively withdrawn from the solution by suctioning the solution volume near the magnet. In particular, in the case where a tubular volume is defined, the gadolinium ligand complex is removed from the tubular volume slowly in the region of the magnet. The purification process may include several cycles of removing volume near the magnet, allowing the gadolinium ligand concentration to increase further, and subsequently removing another volume of gadolinium ligand complex solution.

### Example 4. Locking a Purified Gadolinium Ligand Complex

As described previously, complexes of gadolinium and macrocyclic ligands come in several forms. Generally, the first gadolinium ligand complexes to form are those which contain water molecules in the coordination sphere between gadolinium and the macrocyclic. For example, in the case of DOTA, there are eight sites that can be associated with water. Driving out the water molecules from the coordination sphere of the gadolinium ligand complex takes time and elevated temperature. The present disclosure has found that a temperature of 92° C. to 98° C., preferably about 95° C. applied to a gadolinium ligand solution for at least 24 hours, such as about 32 hours or about 40 hours, is sufficient to drive out all but one of the water molecules. This stable configuration described by the TSA conformation involves seven bonds between the gadolinium ion and the macrocyclic chelate and one bond with a water molecule. The stability of the gadolinium ligand can be increased by removing the final water molecule, causing a transition from the TSA conformation to the TSA′ conformation.

A solution of the gadolinium DOTA complex is too acidic to be of use as an MRI contrast agent. The pH of the solution should be brought up to the pH of blood, in the range of 7.0 to 7.4. This is generally done by the introduction of an amine. Meglumine is used, as illustrated in FIGS. 2A and 2B. Meglumine has a positively charged side associated with the amine group and a negatively charged side associated with hydroxyl groups. When a solution of gadolinium DOTA complex is held, preferably for approximately 2 hours, and preferably at approximately 40° C., in the presence of meglumine, the water molecule is driven away, the hydroxyl group of the meglumine takes the position of the removed water molecule, and the gadolinium DOTA complex is essentially locked in place. Gadolinium ion is sterically hindered from leaving the macrocyclic cage by the presence of the meglumine, and the transition from the stable TSA configuration to the locked in TSA′ configuration is achieved.

This TSA′ structure cannot be obtained through sterilization (121° C.) alone nor will sterilization impact the properly formed TSA′ structure.

### Example 5. Continuous Throughput Purification of Gadolinium Ligand Solutions

A gadolinium ligand solution cooled below the Curie temperature can be passed into a bifurcating conduit. Perpendicular to the conduit, a magnetic field gradient can be established which will clump or aggregate and draw gadolinium ligand complex toward one of the conduit bifurcation arms and away from the other. The result is a continuous method for separating gadolinium ligand complex from contaminating metal-ligand complex. The output side of the contaminating metal-ligand complex arm of the conduit bifurcation can be directed to the input side of the non-bifurcated conduit (upstream) and the solution can be further purified employing multiple passes of a given liquid volume. In a continuous embodiment, the input side of the conduit can also include the addition of new solution to make up for the volume loss on the purified gadolinium ligand side of the conduit bifurcation.

### Example 6. A Method of Measuring Gadolinium Ligand Stability

When gadolinium ligand is introduced into the body the complex encounters a variety of amine containing moieties which act as chelation challenges, and which can destabilize a gadolinium ligand complex causing the irreversible release of free gadolinium ion into the body. Since free gadolinium ion in the body is desired to be avoided, by setting free gadolinium ion concentration levels in the bottle as part of labeling, this measure of free dissociation in water is not relevant to what happens in the body. In particular, free dissociation by water does not challenge metastable forms of gadolinium ligand complex, giving a false impression of their safety. The present method is intended to provide a more relevant safety standard, wherein it can be demonstrated that a gadolinium ligand complex containing fewer than two water molecules is significantly more stable than other metastable forms of gadolinium ligand complex. The method comprises placing the gadolinium ligand solution to be tested in the presence of an ionic chelator, either localized on a gel or separated from the test solution by a semipermeable membrane. The procedure is essentially the same as the purification method of Example 1, except, the goal of this method is to quantify the stability of the gadolinium ligand solution.

For example, a stability measure can be obtained by exposing the gadolinium ligand solution to the chelate challenge for a given period of time and at a given temperature. Then the stability can be measured as the number of gadolinium ions collected by the challenging chelate after the given exposure time. Quantification of the stability measure given in gadolinium ions collected in a standard time can be performed by several methods known in the art. For example, the gadolinium ions can be released from the challenging chelate and quantified by a number of methods, including ICP-mass spec. Alternatively, the concentration of ligand in the gadolinium ligand solution can be compared before and after challenge. Since any gadolinium ion collected due to this method would result in an equal number of gadolinium ligand ions being removed from the test solution. The concentration of gadolinium ligand in the test solution can be quantified using HPLC.

### Example 7. Purification of Twisted Square Antiprismatic (TSA′) Locked Structure

A detailed example of purification process related to the present disclosure is set forth below:

1. A solution is prepared using the TSA′ process disclosure above and comprised of: gadoterate meglumine (Gadoteric acid (organic acid DOTA chelated with Gd3+-Meglumine salt)) having a Composition: 98% gadoterate meglumine with only zero to one water molecule in inner sphere and with the twisted square antiprismatic (TSA′) twist lock structure, 2% imperfect structures (gadoterate meglumine with multiple water molecules, non-twisted square antiprismatic (TSA′) structures, and other non-gadoterate meglumine structures.

2. A cation exchange resin solution is prepared as follows: Optionally, the resin can be conditioned by placing on a fritted glass funnel and washing with deionized water until the filtrate had a pH of 6-7 and a color of <5 APHA.

A BioRad AG50W-X8 or AG50W-X12, respectively, analytical grade (AG) strong cation or ion exchange chromatography resin is converted to the meglumine+ form as follows: To a stirred slurry of about 135 g of washed ion-exchange resin in its protonated form in 405 g of deionized water in a 1 L beaker, meglumine was added in portions, and after a few minutes of stirring the pH of the liquid was measured. When a pH of at least 11 was maintained for 30 min after the last addition of meglumine, stirring was stopped, the liquid was decanted from the resin, deionized water (400 g) was added to reslurry, then the resin was filtered off and washed on the filter with deionized water until the filtrate had a pH of 6-7 and a color of <5 APHA. The resin, now in its meglumine form, was briefly sucked dripless and transferred to a bottle for storage.

3. The initial solution is challenged with the cation exchange resin as follows: The gadoterate meglumine solution in Step 1 of this example above is mixed with 0.9-1.1 equivalents of the prepared resin and then mixed for 20-120 minutes at 15-40 C. The resin will preferentially dechelate imperfect structures (gadoterate meglumine with multiple water molecules, non-twisted square antiprismatic) and sequester the released Gd3+ ions as well as protonated DOTA. For each Gd+3 molecule sequestered, three meglumine+molecules will be released into the solution.
4. The treated solution is separated from the resin using filtration or decantation to isolate the liquid phase
5. The solution composition is adjusted by measuring the meglumine concentration and pH, then adding, as necessary, free base meglumine to return the pH to 7.2-7.4
6. The formulation is fine-tuned as follows:

A metastable test is used to quantify ideal twisted square antiprismatic (TSA′) locked structures (TSA′ specification is greater than 99.5%). If necessary, steps 3 to 5 are repeated with a fresh resin to further improve purity. Optionally, small amounts of fully formed gadoteric acid to achieve 99.5% ideal structure targets can be added. Optionally, final quality control testing can be performed, for example as per United States Pharmacopeia (USP) specifications.

When the above procedure is applied to the commercially available gadoterate meglumine products on the market, it is noteworthy this process as described generates a solution with a minimum of 99.5% to 99.85% of ideal TSA′ cages (5,000-1500 PPM of non ideal cages). The untreated commercial gadoterate meglumine has only 98% ideal TSA cages (20,000 ppm non-ideal cages). Moreover, when a TSA′ Gd Product is exposed to the same process a 99.98% solution of ideal cage structures is achieved (200 PPM of defective cage structures).

### Higher Meglumine Loading

By exposing the intermediate gadoterate meglumine to the resins described above suitable for use in the present disclosure, a higher quantity of meglumine can be added to the product solution while remaining within the US Pharmacopeia (USP) specifications. This is not possible without the resin treatment step of the present disclosure.

During this step, for each gadolinium that the resin sequesters, it releases three meglumine+ molecules. Without wishing to be bound by a single theory, the extra proton helps lower the pH slightly. Additional meglumine beyond the amount added by the resin, is added in the final pH adjustment step. The ability to add extra meglumine appears to further stabilize the TSA′ locked structure.

### Example 8. Metastable Test Procedure & Results

In the present disclosure, the metastable test procedure is used as a diagnostic tool to quantitatively measure % of metastable lanthanide ion complexes formed by a reaction of the present disclosure. For example, the metastable test may be used to quantify both non-ideal structures (metastables) and the purified twisted square antiprismatic (TSA′) locked structure compounds with zero to one water molecule, i.e., an ideal cage structure, which is generated in a percentage greater than 99.5%.

### General Description of Metastables Test

In one aspect, the present disclosure relates to a method for determining metastable lanthanide ion dodecane tetraacetic acid (DOTA) complex, which comprises steps of (i.e., also identified herein as “The Metastables Test):
A. forming and stirring a mixture of pre-washed polystyrenic macroporous, strong acid cation exchange resin and deionized water for 24 hours at ambient temperature in a flask;
B. adding a solution of lanthanide ion dodecane tetraacetic acid (DOTA), such as gadoterate meglumine, to the mixture with continued stirring at ambient temperature;
C. monitoring reaction progress by taking test samples of the reaction mixture at different reaction time points;
   wherein:
   a. the test samples are analyzed over a range of 250-350 nm by using a UV spectrometer;
   b. 1^{st} sample point taken at t=0 which occurs at the end of gadoterate meglumine addition;
   c. a known generated compound of the reaction mixture is designated as a relative reference standard peak against which newly generated metastable gadolinium complex peaks are compared to obtain corrected absorbance value ratios;
D. plotting the collected corrected absorbance ratios corresponding to test sample versus time to generate a graph;
   wherein on the graph:
      1. reference compound shows a straight line at 100%;
      2. other metastable complexes:
         a. appear above that line if they contain fewer metastable complexes and
         b. below that line if they contain more metastable complexes; and
      3. any decline from 100% at the first time point over the course of the reaction timeline to the end value may be interpreted as the reduction in metastable gadolinium complexes.

Those skilled in the art may also adapt other known conventional analytical methods for determining qualitative or quantitative amounts of lanthanide metal complexes, as taught by the present disclosure.

### Metastable Test Procedure, Data Analysis and Interpretation

To a 20 mL screw-capped vial were added 1.0 g of washed C150 ion-exchange resin, 5.0 g of deionized water, and a magnetic stir bar. The mixture was stirred at ambient temperature for about 24 h. The gadoterate meglumine solution (approx. 0.5 mmol/mL) (5.0 g) was added to the vial as quickly as possible. The end of the addition was taken as t=0. The resulting solution was stirred at ambient temperature.

Samples for analysis by UV were taken at suitable time points, with the first sampling typically at t=1 minute. The samples were filtered with a syringe with a 0.45 μm syringe filter directly into a cuvette for UV data acquisition, and then returned to the vial. Depending on the number of sampling time points, the volume required for UV data acquisition, and quantity of the C150/water/gadoterate meglumine mixture, it might be chosen to not return the filtered sample. If so desired, an aqueous salt solution may be used, such as, but not limited to, sodium chloride, instead of deionized water to modify the swelling state of the resin.

The UV spectra for the time points were acquired over a range of 250-350 nm. The absorbance for the peak at 274 nm was corrected for changes in baseline by linearly interpolating the baseline from before and after the group of peaks around 274 nm (i.e., approximately from 270 to 280 nm) and subtracting the interpolated baseline value at 274 nm from the absorbance of the 274 nm peak maximum.

For the analysis of a time course experiment, it is useful to plot the corrected absorbances of the 274 nm peak as a percentage of the corrected absorbances of the 274 nm peak at the first time point versus time. The decline from 100% at the first time point over the course of the experiment to the end value may be interpreted as the reduction in metastable gadolinium complexes.

A simple way of comparing the content of metastable gadolinium complexes between compounds is then to compare the percentage values at the last time point. In an alternate way, compounds may be compared by designating one compound as the reference and dividing the corrected absorbances of the 274 nm peak at each time point by the corresponding absorbances of the 274 nm peak of the reference. When these ratios are plotted versus time, the reference compound shows a straight line at 100% and the other compounds are above that line if they contain fewer metastable complexes and below that line if they contain more metastable complexes.

Dotarem® without Resin Treatment

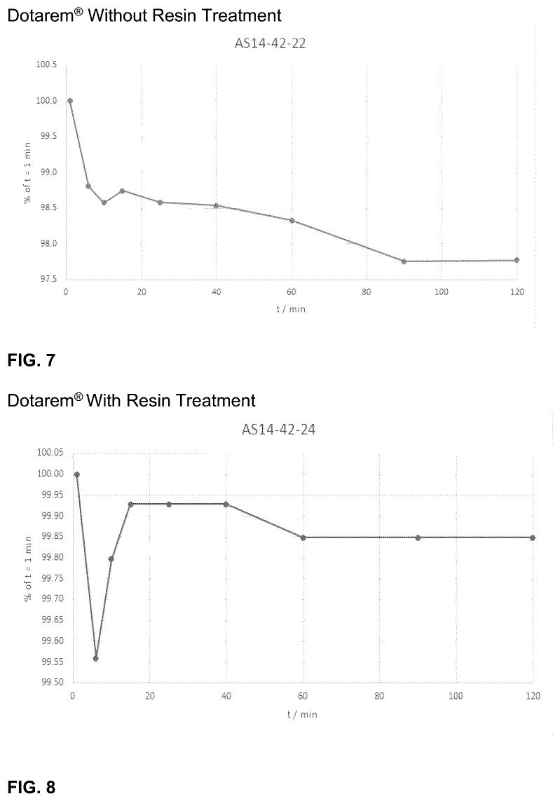

FIG. 7 shows that untreated Dotarem® releases approximately 20,000 ppm Gd+3 during the 120 minute metastables test. This indicates that commercial Dotarem® has 2% of cages that do not have the most stable TSA′ locked structure according to the present disclosure.

The X axis is the gadoterate concentration at each time point divided by initial gadoterate concentration expressed in percent

Dotarem® with Resin Treatment

FIG. 8 shows Dotarem® after treating commercial Dotarem® with the indicated strong acid cation resin, the number of non TSA′ locked structure cages has been reduced by 75-90%, that is, only 2000-5000 ppm of Gd+3 was sequestered in the metastables test vs. 20,000 in the untreated material. These non TSA′ cages were dechelated in the presence of the resin during treatment. The dechelated Gd+3 and the resulting DOTA+were captured by the resin.

The X axis is the gadoterate concentration at each time point divided by initial gadoterate concentration expressed in percent

Gadzero Prior to and after Resin Treatment

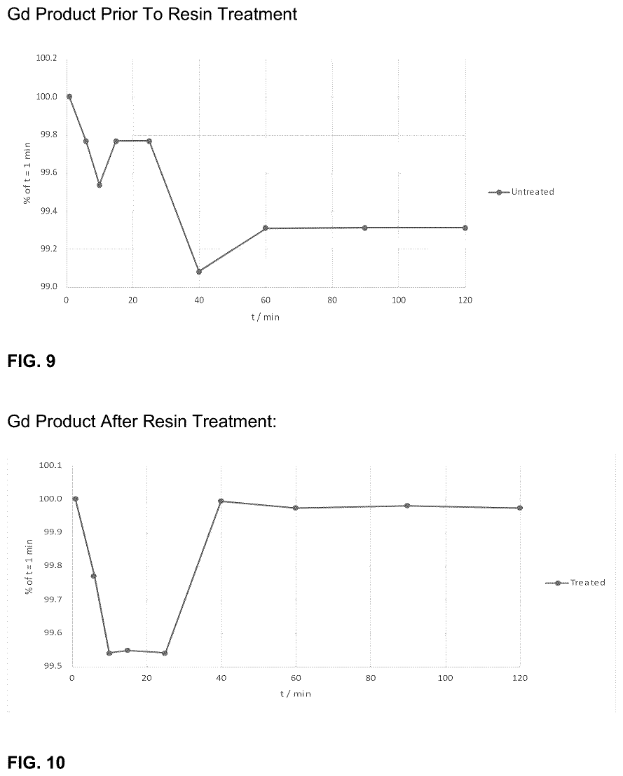

FIG. 9 shows a Gd Product prior to resin treatment and FIG. 10 which shows a Gd Product after resin treatment.

It should be noted that the terms “first”, “second”, and the like are used herein to modify various elements. These modifiers do not imply a spatial, sequential, or hierarchical order to the modified elements unless specifically stated.

As used herein, the terms “a” and “an” mean “one or more” unless specifically indicated otherwise.

As used herein, the term “substantially” means the complete or nearly complete extent or degree of an action, characteristic, property, state, structure, item, or result. The exact allowable degree of deviation from absolute completeness can in some cases depend on the specific context. However, generally, the nearness of completion will be to have the same overall result as if absolute and total completion were obtained.

As used herein, the term “comprising” means including, but not limited to; the term “consisting essentially of” means that the method, structure, or composition includes steps or components specifically recited and may also include those that do not materially affect the basic novel features or characteristics of the method, structure, or composition; and the term “consisting of” means that the method, structure, or composition includes only those steps or components specifically recited.

As used herein, the term “about” is used to provide flexibility to a numerical range endpoint by providing that a given value can be “a little above” or “a little below” the endpoint. Further, where a numerical range is provided, the range is intended to include any and all numbers within the numerical range, including the end points of the range.

As used herein, the term “substantially free of metastable lanthanide complexes and free lanthanide ions” means metastable lanthanide complexes and free lanthanide ions are undetectable by diagnostic testing methods as of the time of the present disclosure.

The term “chelating agent” refers to ligands that form metal coordination complexes that contain a plurality of metal donor atoms arranged so that 5, 6, or 7-membered coordination rings are obtained. Coordination rings preferably have an uncoordinated backbone of carbon atoms or uncoordinated heteroatoms linking donor metal atoms. Chelating agents are chemical compounds that coordinate with metal ions to form a stable, water-soluble complex. Chelating agents are also known as chelants, chelators, or sequestering agents. Chelating agents have a ring-like center which forms at least two bonds with the metal ion allowing it to be excreted. As used herein, a chelating agent has at least two functional groups which donate a pair of electrons to the metal, such as ═O, —NH₂ or —COO—. Furthermore, these groups must be located so as to allow coordination with the metal.

The term “macrocyclic” has its ordinary and customary meaning in the field of coordination chemistry and refers to a chelator in which at least some of the chelator's metal donor atoms are bonded as part of a ring system.

The expression “chelator in uncomplexed form” refers to “free chelator”, that is, without any coordinated metal ions. A chelator in uncomplexed form does not contain any coordinated lanthanide or other metal ions and is thus fully available for subsequent metal complex formation. The “chelator in uncomplexed form” may contain metal ions in ionic form, for example, when it is present as salts of a metal donor group, for example, a carboxylic acid.

The expression “metastable lanthanide complex” refers to a lanthanide complex wherein the metal ion is not fully contained within the coordination ring. “Metastable lanthanide complex” has a less stable kinetic stability constant compared to stable or fully coordinated lanthanide complexes.

The term “suitable cation exchange resins” such as those materials used in purifications described herein are cation exchange resins, which are insoluble polymers with positively charged functional groups that help exchange pollutant cations with less objectionable cations. As conventionally understood in the art, cation exchange resins are deployed in ion exchange systems to specifically extract cationic impurities. Also see Definition Section in this application.

Examples of suitable cation exchange resins for use in the present disclosure include highly purified, analytical, biotech, molecular biology grade (AG) ion exchange chromatography resins, which are extensively purified to remove both organic and inorganic impurities, sized to consistently give narrow wet mesh ranges, which provide high resolution and excellent reproducibility. Such AG resins are available as both strong and weak anion and cation exchangers and as mixed-bed ion exchangers. Many are available in several ionic forms and can be converted from one form to another. AG resins are primarily used for the separation of low molecular weight compounds such as inorganic ions, organic acids, nucleic acids, or carbohydrates.

Examples of ion exchange resins suitable for use in the present disclosure may include, but are not limited to products identified by or manufactured by: Bio-Rad Laboratories, Inc., such as those identified as biotech, molecular biological or analytical grade resins: AG50W-X8, AG50W-X12 and AG50W-X16; Dowex™ 50WX8 (spherical fine mesh resin microporous copolymer of styrene and divinylbenzene (DVB) that results in maximum resistance to oxidation, reduction, mechanical wear and breakage) and the like, respectively of ionic hydrogen form, varying percentages of cross-linking, mesh and bead sizes (i.e., e.g., for details see generally www.bio-rad.com and Analytical Grade Ion Exchange Resins IBio-Rad (i.e., see www.bio-rad.com/en-us/category/analytical-grade-ion-exchange-resins?ID=6512cba2-aOc6-4062-915c-6d0df409bdec).

### Bio-Rad Resin Specification Information Suitable for Use in Present Disclosure

Specifications of AG ®50 Resin

| | | MW | Stability/pH | | | |
|---|---|---|---|---|---|---|
| | | Exclusion | Range | meq/ml) | (g/ml) | |
| Type: Strong Cation | 2% | 2,700 | 0-14/0-14 | 0.6 | 0.7 | Hydrogen |
| Functional Group R- | 4% | 1,400 | | 1.1 | 0.8 | Hydrogen |
| SO3— | | | | | | |
| Matrix: Styrene | 8% | 1,000 | | 1.7 | 0.8. | Hydrogen |
| divinylbenzene | 12% | 400 | | 2.1 | 085 | Hydrogen |

Applications: Lower crosslinkage resins are used for separation or concentration of peptides, nucleotides, and amino acids. Higher crosslinkage resins are useful for separations of smaller peptides and amino acids, removal of cation, and separation of metal ions.

AG® 50W resins suitable for use in the present disclosure are microporous strong acid cation resins. Such analytical Grade AG® 50W resin has been sized, purified, and converted to make it suitable for accurate, reproducible purification. It is expected that nonanalytical grade versions of the same resins could be used with a modification of the process parameters used in the present disclosure. AG® 50W resin is composed of sulfonic acid functional groups attached to styrene divinylbenzene copolymer lattice.

The amount of resin crosslinking determines the bead pore size. The X8 and X12 versions are relatively highly crosslinked. A resin with a higher crosslinkage has a tighter structure that is less permeable to higher molecular weight substances. It also has a higher physical resistance to shrinking and swelling, so it absorbs less water and swells to a lesser degree than low-crosslinked resins.

More particularly, AG® 50W resins are microporous strong acid cation resins. The MW cut off for the X8 and X12 resins are 1000 and 400, respectively. The Analytical Grade AG® 50W resin has been sized, purified, and converted to make it suitable for accurate, reproducible purification. It is expected that non analytical grade versions of the same resins could be used with a modification of the process parameters. AG® 50W resin is composed of sulfonic acid functional groups attached to styrene divinylbenzene copolymer lattice. There is one sulfonic acid group in each crosslinked styrene molecule. The amount of resin crosslinking determines the bead pore size. The X8 and X12 versions are relatively highly crosslinked (8% and 12%, respectively). A resin with a higher crosslinkage has a tighter structure that is less permeable to higher molecular weight substances. It also has a higher physical resistance to shrinking and swelling, so it absorbs less water and swells to a lesser degree than low crosslinked resins. Without wishing to be bound by theory, some or all of the AG® 50W attributes are believed to be critical in obtaining the highest percentage of TSA′ locked structures.

The table above shows the approximate molecular weight exclusion limits in water for resins of various crosslinkages. Some or all of the AG® 50W attributes are critical in obtaining the highest percentage of TSA′ locked structure cages. Strong acid cation resins other than the AG® 50W resins described above have been tested for use in the present disclosure, but results were found not as favorable when other resins, including different grades of microporous or microporous resins with similar or differing chemistries were used. In some cases, the final percentage of TSA′ ideal cages was lower, while in other cases the benefits of this resin treatment were lost over time, i.e. the equilibrium of the product shifted back to the original metastables levels.

In one aspect or embodiment, the present disclosure relates to a method of making a lanthanide ion dodecane tetraacetic acid (DOTA) complex contrast agent, which comprises:
adding DOTA to purified water in a vessel to form a solution or suspension; stirring the solution or suspension while heating a temperature of the solution or suspension to 92° C. to 98° C.;
adding lanthanide series oxide incrementally to the solution or suspension, wherein each incremental addition is made when the solution or suspension from a previous addition turns substantially clear; and
repeating the adding until a substantially equimolar amount of lanthanide series oxide is added as compared to DOTA, thus forming a lanthanide oxide DOTA solution.

In another aspect or embodiment, the present disclosure relates to a method further which comprises:
reacting the lanthanide oxide DOTA solution at 92° C. to 98° C. for at least 24 hours to obtain a lanthanide oxide DOTA complex having a TSA) structure.

In another aspect or embodiment, the present disclosure relates to a method where the lanthanide series oxide is gadolinium oxide.

In another aspect or embodiment, the present disclosure relates to further comprising: cooling the lanthanide oxide DOTA solution to 35° C. to 45° C.; and adding meglumine or a meglumine like compound to the cooled lanthanide oxide DOTA solution to obtain a lanthanide oxide DOTA complex having a TSA′ structure.

In another aspect or embodiment, the present disclosure relates to a method of purifying lanthanide metal ligand complexes comprising:
dissolving a lanthanide metal ligand complex in a solvent to obtain a lanthanide metal ligand solution;
placing the lanthanide metal ligand solution in a first side of a chamber and a solvent in a second side of the chamber, wherein the chamber is partitioned into the first and second side by a semipermeable membrane;
applying a static magnetic field sufficient to cause the lanthanide metal ligand complex to aggregate;
lowering a temperature of the aggregated lanthanide metal ligand solution to below a Curie temperature of the lanthanide metal ligand complex; and removing the aggregated lanthanide metal ligand solution from the first side.

In another aspect or embodiment, the present disclosure relates to a method where the first and second side contain an electrolyte, and an electric field is applied to the chamber.

In another aspect or embodiment, the present disclosure relates to a method, where the semipermeable membrane has an average porosity in a range of 1 to 50 kDa.

In another aspect or embodiment, the present disclosure relates to a method, where the second side contains an ionic gel suitable for capturing metal ions, and free metal ions are removed from the first side and captured by the ionic gel in the second side.

In another aspect or embodiment, the present disclosure relates to a method, where more than 99% mol of free metal ion and non-ferromagnetic metal ligand complex is removed from the first side to the second side.

In another aspect or embodiment, the present disclosure relates to a lanthanide series ion dodecane tetraacetic acid (DOTA) MRI contrast agent which comprises:
a plurality of lanthanide ions; and
a plurality of DOTA chelate molecules, wherein the plurality of lanthanide ions and the plurality of DOTA chelate molecules form lanthanide ion DOTA complexes, wherein the lanthanide ion DOTA complexes are in a TSA structure having only one coordinated water molecule.

In another aspect or embodiment, the present disclosure relates to an MRI contrast agent in which the lanthanide series ion is Gd³⁺.

In another aspect or embodiment, the present disclosure relates to an MRI contrast agent, where greater than 95% of the complexes of gadolinium and DOTA possess a TSA structure.

In another aspect or embodiment, the present disclosure relates to an MRI contrast agent of lanthanide ion-dodecane tetraacetic acid (DOTA) complexes which comprises:
a plurality of lanthanide ions;
a plurality of DOTA chelate molecules; and
meglumine or a meglumine-like compound,
where the lanthanide ion-DOTA complexes comprise TSA′ structure and having no coordinated water molecule.

In another aspect or embodiment, the present disclosure relates to a MRI contrast agent, where the plurality of lanthanide ions consist essentially of Gd³⁺.

In another aspect, the present disclosure relates to a MRI contrast agent, where the lanthanide ion-DOTA complexes consist of a TSA′ structure.

In another aspect or embodiment, the present disclosure relates to method for making a lanthanide ion dodecane tetraacetic acid (DOTA) complex contrast agent which comprises steps of:
(1) adding an amount of 1,4,7,10-tetraazacyclo-dodecane-N, N′, N″, N′″-tetraacetic acid (DOTA) to purified water in a vessel to form a solution or suspension;
(2) stirring and simultaneously heating the solution or suspension to a temperature of at least 90° C.;
(3) adding a lanthanide series oxide incrementally controlled to the stirring solution or suspension;
   where:
      each subsequent addition of the lanthanide series oxide occurs after the solution or suspension turns to a substantially clear color;
(4) repeating step (3) until a substantially equimolar amount of the lanthanide series oxide is added relative or comparative to the amount of DOTA to form a lanthanide oxide DOTA solution or suspension;
(5) reacting the lanthanide oxide DOTA solution or suspension formed in step (4) at a temperature of at least about 90° C. for at least 24 hours to obtain a lanthanide oxide DOTA complex having a twisted square antiprismatic (TSA) structure
(6) cooling the lanthanide oxide DOTA solution temperature to at least about 30° C.; and
(7) adding free base meglumine to the cooled lanthanide oxide DOTA solution or suspension to form in at least 95 to 98% or greater yield a lanthanide oxide DOTA complex having a twisted square antiprismatic (TSA′) structure;
   where:
      the twisted square antiprismatic (TSA′) structure is maintained at a pH of at least about 7.

In another aspect or embodiment, the present disclosure relates to a method for making a lanthanide ion dodecane tetraacetic acid (DOTA) complex contrast agent which comprises steps of:
(1) adding an amount of 1,4,7,10-tetraazacyclo-dodecane-N, N′, N″, N′″-tetraacetic acid (DOTA) to purified water in a vessel to form a solution or suspension;
(2) stirring and simultaneously heating the solution or suspension to a temperature range between at least about 92° C. to about 98° C.;
(3) adding a lanthanide series oxide incrementally controlled to the stirring solution or suspension;
   where:
      each subsequent addition of the lanthanide series oxide occurs after the solution or suspension turns to a substantially clear color;
(4) repeating step (3) until a substantially equimolar amount of the lanthanide series oxide is added relative or comparative to the amount of DOTA previously added in step (1) to form a lanthanide oxide DOTA solution or suspension;
(5) reacting the lanthanide oxide DOTA solution formed in step (4) at a temperature of about 92° C. to 98° C. for at least 32 hours to obtain a lanthanide oxide DOTA complex having a twisted square antiprismatic (TSA) structure
(6) cooling the lanthanide oxide DOTA solution temperature of about 35° C. to 45° C.; and
(7) adding free base meglumine to the cooled lanthanide oxide DOTA solution to form in at least 99.5% yield a lanthanide oxide DOTA complex having a twisted square antiprismatic (TSA′) structure;
   where:
      the twisted square antiprismatic (TSA′) structure is maintained in a pH in a range between about 7.2 to 7.4.

In another aspect or embodiment, the present disclosure relates to a method for making a lanthanide ion dodecane tetraacetic acid (DOTA) complex contrast agent, further which comprises additional purification steps of:
taking:
   the at least 95 to 98% as defined in claim 16 or 99.5% yield a lanthanide oxide DOTA complex having the twisted square antiprismatic (TSA′) structure, a product formed in the solution or a suspension of step (7); and
adding:
   at least 0.9 to 1.1 equivalents of a strong acid cation exchange resin; and de-ionized water;
to form a combined new mixture solution or suspension that is stirred in the flask for at least an additional 20 minutes to 2 hours at a temperature between about 15° C. to 40° C.;
   where:
      the strong acid cation exchange resin assists in removing additional formed products and other residual materials in the new mixture solution or suspension by:
      dechelating non-ideal cages (non twisted square antiprismatic (TSA′) structures;
      sequestering any released Gd³⁺ ions; and/or
      protonated DOTA;
      where:
         for each Gd³⁺ molecule sequestered, three meglumine+molecules is released into the new mixture solution or suspension;
filtering the combined new mixture solution or suspension of step (8) to separate the strong acid cation exchange resin from and isolate the further purified product:
   lanthanide oxide DOTA complex having a twisted square antiprismatic (TSA′) structure of step (7)
   in the solution or suspension;
adjusting and fine-tuning the solution and suspension by:
   measuring the meglumine concentration and pH
   adjusting pH back to about 7.2-7.4 by adding free base meglumine;
performing analytical tests to quantify percentage of the further purified product of step (10);
repeating steps (8) to (10) to further improve purity of the product, if necessary;
performing final quality control testing as per US Pharmacopeia (USP); and
   yielding a highly purified solution or suspension with at least 99.5% twisted square antiprismatic (TSA′) structure.

In another aspect or embodiment, the present disclosure relates to a process for
making lanthanide ion dodecane tetraacetic acid (DOTA) complex contrast agent formed by:
   (1) adding an amount of 1,4,7,10-tetraazacyclo-dodecane-N, N′, N″, N′″-tetraacetic acid (DOTA) to purified water in a vessel to form a solution or suspension;
   (2) stirring and simultaneously heating the solution or suspension; and
   (3) adding a lanthanide series oxide to the stirring solution or suspension;
where the improvement comprises steps of:
   (4) controlling each subsequent incremental addition of the lanthanide series oxide until after the solution or suspension turns to a substantially clear color;
   (5) repeating step (4) until a substantially equimolar amount of the lanthanide series oxide is added relative or comparative to the amount of DOTA previously added in step (1) to form a lanthanide oxide DOTA solution or suspension;
   (6) heating the lanthanide oxide DOTA solution or suspension formed in step (4) to a temperature of at least about 90° C. for at least 24 hours to 32 hours to form a lanthanide oxide DOTA complex having a twisted square antiprismatic (TSA) structure;
   (7) cooling the lanthanide oxide DOTA solution or suspension temperature to at least about 30° C. to 40° C.; and
   (8) adding free base meglumine to the cooled lanthanide oxide DOTA solution to:
      form in at least 95 to 98% or greater yield a lanthanide oxide DOTA complex having a twisted square antiprismatic (TSA′) structure; and
      where:
         the twisted square antiprismatic (TSA′) structure is maintained at a pH of at least about 7.

In another aspect or embodiment, the present disclosure relates to a process for making lanthanide ion dodecane tetraacetic acid (DOTA) complex contrast agent formed by:
(1) adding an amount of 1,4,7,10-tetraazacyclo-dodecane-N, N′, N″, N′″-tetraacetic acid (DOTA) to purified water in a vessel to form a solution or suspension;
(2) stirring and simultaneously heating the solution or suspension; and
(3) adding a lanthanide series oxide to the stirring solution or suspension;
   where the improvement comprises steps of:
(4) controlling each subsequent incremental addition the lanthanide series oxide until after the solution or suspension turns to a substantially clear color;
(5) repeating step (4) until a substantially equimolar amount of the lanthanide series oxide is added relative or comparative to the amount of DOTA previously added in step (1) to form a lanthanide oxide DOTA solution or suspension;
(6) heating the lanthanide oxide DOTA solution or suspension formed in step (4) to a temperature of at least about about 92° C. to 98° C. for at least 32 hours to form a lanthanide oxide DOTA complex having a twisted square antiprismatic (TSA) structure;
(7) cooling the lanthanide oxide DOTA solution or suspension temperature to at least about 35° C. to 45° C.; and
(8) adding free base meglumine to the cooled lanthanide oxide DOTA solution to:
   form in at least 99.5% or greater yield a lanthanide oxide DOTA complex having a twisted square antiprismatic (TSA′) structure; and
   wherein:
      the twisted square antiprismatic (TSA′) structure is maintained at a pH of between 7.2 to 7.4.

In another aspect or embodiment, the present disclosure relates to a process for making lanthanide ion dodecane tetraacetic acid (DOTA) complex contrast agent formed by steps defined according to the present disclosure, where the improvement further comprises additional purification improvement steps of:
(9) taking:
   the at least 95 to 98% or 99.5% yield a lanthanide oxide DOTA complex having the twisted square antiprismatic (TSA′) structure, a product formed in the solution or a suspension of step (7); and
   adding:
   at least 0.9 to 1.1 equivalents of a strong acid cation exchange resin; and de-ionized water;
   to form a combined new mixture solution or suspension that is stirred in the flask for at least an additional 20 minutes to 2 hours at a temperature between about 15° C. to 40° C.;
   where:
   the strong acid cation exchange resin assists in removing additional formed products and other residual materials in the new mixture solution or suspension by:
   dechelating non-ideal cages (non twisted square antiprismatic (TSA′) structures;
   sequestering any released Gd³⁺ ions; and/or protonated DOTA;
      where:
         for each Gd³⁺ molecule sequestered, three meglumine+molecules is released into the new mixture solution or suspension;
(10) filtering the combined new mixture solution or suspension of step (8) to separate the strong acid cation exchange resin from and isolate the further purified product:
   lanthanide oxide DOTA complex having a twisted square antiprismatic (TSA′) structure of step (7)
      in the solution or suspension;
(11) adjusting and fine-tuning the solution and suspension by:
   measuring the meglumine concentration and pH
   adjusting pH back to about 7.2-7.4 by adding free base meglumine;
(12) performing analytical tests to quantify percentage of the further purified product of step (10);
(13) repeating steps (8) to (10) to further improve purity of the product, if necessary;
(14) performing final quality control testing as per US Pharmacopeia (USP); and
(15) yielding a highly purified solution or suspension with at least 99.5% twisted square antiprismatic (TSA′) structure.

In another aspect or embodiment, the present disclosure relates to a process for making lanthanide ion dodecane tetraacetic acid (DOTA) complex contrast agent formed by:
(1) adding an amount of 1,4,7,10-tetraazacyclo-dodecane-N, N′, N″, N′″-tetraacetic acid (DOTA) to purified water in a vessel to form a solution or suspension;
(2) stirring and simultaneously heating the solution or suspension; and
(3) adding a lanthanide series oxide to the stirring solution or suspension;
   where the improvement comprises purification improvement steps of:
(4) controlling each subsequent incremental addition of the lanthanide series oxide until after the solution or suspension turns to a substantially clear color;
(5) repeating step (4) until a substantially equimolar amount of the lanthanide series oxide is added relative or comparative to the amount of DOTA previously added in step (1) to form a lanthanide oxide DOTA solution or suspension;
(6) heating the lanthanide oxide DOTA solution or suspension formed in step (4) to a temperature of at least about 90° C. for at least 24 hours to 32 hours to form a lanthanide oxide DOTA complex having a twisted square antiprismatic (TSA) structure;
(7) cooling the lanthanide oxide DOTA solution or suspension temperature to at least about 30° C. to 40° C.; and
(8) adding free base meglumine to the cooled lanthanide oxide DOTA solution to:
   form in at least 95 to 98% or greater yield a lanthanide oxide DOTA complex having a twisted square antiprismatic (TSA′) structure; and
      where:
         the twisted square antiprismatic (TSA′) structure is maintained at a pH
         of at least about 7;
(9) taking:
   the at least 95 to 98% as defined in claim 19 or 99.5% as defined in claim 20 yield a lanthanide oxide DOTA complex having the twisted square antiprismatic (TSA′) structure, a product formed in the solution or a suspension of step (7); and
      adding:
   at least 0.9 to 1.1 equivalents of a strong acid cation exchange resin; and de-ionized water;
      to form a combined new mixture solution or suspension that is stirred in the flask for at least an additional 20 minutes to 2 hours at a temperature between about 15° C. to 40° C.;
   where:
      the strong acid cation exchange resin assists in removing additional formed products and other residual materials in the new mixture solution or suspension by:
      dechelating non-ideal cages (non-twisted square antiprismatic (TSA′) structures;
      sequestering any released Gd³⁺ ions; and/or protonated DOTA;
      wherein:
         for each Gd³⁺ molecule sequestered, three meglumine+molecules is released into the new mixture solution or suspension;
(10) filtering the combined new mixture solution or suspension of step(8) to separate the strong acid cation exchange resin from and isolate the further purified product:
   lanthanide oxide DOTA complex having a twisted square antiprismatic (TSA′) structure of step (7)
      in the solution or suspension;
(11) adjusting and fine-tuning the solution and suspension by:
   measuring the meglumine concentration and pH; and
   adjusting pH back to about 7.2-7.4 by adding free base meglumine;
(12) performing analytical tests to quantify the percentage of the further purified product of step (10);
(13) repeating steps (8) to (10) to further improve the purity of the product, if necessary;
(14) performing final quality control testing as per US Pharmacopeia (USP); and
(15) yielding a highly purified solution or suspension with at least 99.5% twisted square antiprismatic (TSA′) structure.

Without wishing to be bound by theory, it is believed that the absence of inner-sphere water molecules can significantly affect the stability of a metal complex. Complexes that lack inner-sphere water molecules are generally more stable towards dissociation and oxidation at neutral pH. This increased stability is advantageous for certain applications, such as when designing metal complex probes that prioritize inertness and stability over other factors. Even in the absence of inner-sphere water molecules, substantial interactions can occur through outer-sphere water molecules. These interactions can contribute to the overall stability and functionality of the complex, especially if the complex has donor groups like amides or alcohols that can form hydrogen bonds with water. For MRI contrast agents, the relaxivity (r₁) is influenced by the presence of inner-sphere water molecules. However, if the design prioritizes stability and inertness, a closed coordination sphere without inner-sphere water molecules can be advantageous. The absence of inner-sphere water molecules means that the complex relies more on second-sphere interactions, which can still provide significant paramagnetic shifts and relaxivity.

The above summary is not intended to describe each disclosed implementation, as features in this disclosure can be incorporated into additional features as detailed herein below unless clearly stated to the contrary.

While the present disclosure has been described with reference to one or more exemplary embodiments, it will be understood by those skilled in the art, that various changes can be made, and equivalents can be substituted for elements thereof without departing from the scope of the present disclosure. In addition, many modifications can be made to adapt a particular situation or material to the teachings of the present disclosure without departing from the scope thereof. The various references to journals, patents, and other publications which are cited herein comprise the state of the art and are incorporated herein by reference as though fully set forth. Therefore, it is intended that the present disclosure will not be limited to the particular embodiments disclosed herein, but that the disclosure will include all aspects falling within the scope of a fair reading of appended claims. It is to be understood that the disclosure is not limited to the embodiments illustrated hereinabove and the right is reserved to the illustrated embodiments and all modifications coming within the scope of the following claims.

What is claimed is:

1. A method of making a lanthanide ion dodecane tetraacetic acid (DOTA) complex contrast agent comprising:
adding DOTA to purified water in a vessel to form a solution or suspension;
stirring the solution or suspension while heating a temperature of the solution or suspension to 92° C. to 98° C.;
adding lanthanide series oxide incrementally to the solution or suspension, wherein each incremental addition is made when the solution or suspension from a previous addition turns substantially clear; and
repeating the adding until a substantially equimolar amount of lanthanide series oxide is added as compared to DOTA, thus forming a lanthanide oxide DOTA solution.

2. The method according to claim 1, further comprising:
reacting the lanthanide oxide DOTA solution at 92° C. to 98° C. for at least 24 hours to obtain a lanthanide oxide DOTA complex having a TSA structure.

3. The method according to claim 1, wherein the lanthanide series oxide is gadolinium oxide.

4. The method according to claim 2, further comprising:
cooling the lanthanide oxide DOTA solution to 35° C. to 45° C.; and
adding meglumine or a meglumine like compound to the cooled lanthanide oxide DOTA solution to obtain a lanthanide oxide DOTA complex having a TSA′ structure.

5. A method of purifying lanthanide metal ligand complexes comprising:
dissolving a lanthanide metal ligand complex in a solvent to obtain a lanthanide metal ligand solution;
placing the lanthanide metal ligand solution in a first side of a chamber and a solvent in a second side of the chamber, wherein the chamber is partitioned into the first and second side by a semipermeable membrane;
applying a static magnetic field sufficient to cause the lanthanide metal ligand complex to aggregate;
lowering a temperature of the aggregated lanthanide metal ligand solution to below a Curie temperature of the lanthanide metal ligand complex; and
removing the aggregated lanthanide metal ligand solution from the first side.

6. The method according to claim 5, wherein the first and second side contain an electrolyte, and an electric field is applied to the chamber.

7. The method according to claim 5, wherein the semipermeable membrane has an average porosity in a range of 1 to 50 kDa.

8. The method according to claim 5, wherein the second side contains an ionic gel suitable for capturing metal ions, and free metal ions are removed from the first side and captured by the ionic gel in the second side.

9. The method according to claim 5, wherein more than 99% mol of free metal ion and non-ferromagnetic metal ligand complex is removed from the first side to the second side.

10. A lanthanide series ion dodecane tetraacetic acid (DOTA) MRI contrast agent comprising:
a plurality of lanthanide ions; and
a plurality of DOTA chelate molecules, wherein the plurality of lanthanide ions and the plurality of DOTA chelate molecules form lanthanide ion DOTA complexes, wherein the lanthanide ion DOTA complexes are in a TSA structure having only one coordinated water molecule.

11. The MRI contrast agent according to claim 10, wherein the lanthanide series ion is Gd³⁺.

12. The MRI contrast agent according to claim 11, wherein greater than 95% of the complexes of gadolinium and DOTA possess a TSA structure.

13. An MRI contrast agent of lanthanide ion-dodecane tetraacetic acid (DOTA) complexes comprising:
a plurality of lanthanide ions;
a plurality of DOTA chelate molecules; and
meglumine or a meglumine like compound,
wherein the lanthanide ion-DOTA complexes comprise TSA′ structure and having no coordinated water molecule.

14. The MRI contrast agent, according to claim 13, wherein the plurality of lanthanide ions consists essentially of Gd³⁺.

15. The MRI contrast agent according to claim 13, wherein the lanthanide ion-DOTA complexes consist of a TSA′ structure.

16. A method for making a lanthanide ion dodecane tetraacetic acid (DOTA) complex contrast agent comprising steps of:
(1) adding an amount of 1,4,7,10-tetraazacyclo-dodecane-N, N′, N″, N′″-tetraacetic acid (DOTA) to purified water in a vessel to form a solution or suspension;
(2) stirring and simultaneously heating the solution or suspension to a temperature of at least 90° C.;
(3) adding a lanthanide series oxide incrementally controlled to the stirring solution or suspension;
wherein:
each subsequent addition of the lanthanide series oxide occurs after the solution or suspension turns to a substantially clear color;
(4) repeating step (3) until a substantially equimolar amount of the lanthanide series oxide is added relative or comparative to the amount of DOTA to form a lanthanide oxide DOTA solution or suspension;
(5) reacting the lanthanide oxide DOTA solution or suspension formed in step (4) at a temperature of at least about 90° C. for at least 24 hours to obtain a lanthanide oxide DOTA complex having a twisted square antiprismatic (TSA) structure
(6) cooling the lanthanide oxide DOTA solution temperature to at least about 30° C.; and
(7) adding free base meglumine to the cooled lanthanide oxide DOTA solution or suspension to form in at least 95 to 98% or greater yield a lanthanide oxide DOTA complex having a twisted square antiprismatic (TSA′) structure;
wherein:
the twisted square antiprismatic (TSA′) structure is maintained at a pH of at least about 7.

17. The method for making a lanthanide ion dodecane tetraacetic acid (DOTA) complex contrast agent according to claim 16, further comprising additional purification steps of:
(8) taking:
the at least 95 to 98% yield a lanthanide oxide DOTA complex having the twisted square antiprismatic (TSA′) structure, a product formed in the solution or a suspension of step (7); and
adding:
at least 0.9 to 1.1 equivalents of a strong acid cation exchange resin; and
de-ionized water;
to form a combined new mixture solution or suspension that is stirred in the flask for at least an additional 20 minutes to 2 hours at a temperature between about 15° C. to 40° C.;
wherein:
the strong acid cation exchange resin assists in removing additional formed products and other residual materials in the new mixture solution or suspension by: dechelating non-ideal cages (non twisted square antiprismatic (TSA′) structures; sequestering any released Gd³⁺ ions; and/or protonated DOTA;
wherein: for each Gd³⁺ molecule sequestered, three meglumine+molecules is released into the new mixture solution or suspension;
(9) filtering the combined new mixture solution or suspension of step (8) to separate the strong acid cation exchange resin from and isolate the further purified product: lanthanide oxide DOTA complex having a twisted square antiprismatic (TSA′) structure of step (7)
in the solution or suspension;
(10) adjusting and fine-tuning the solution and suspension by:
a. measuring the meglumine concentration and pH
b. adjusting pH back to about 7.2-7.4 by adding free base meglumine;
(11) performing analytical tests to quantify percentage of the further purified product of step (10);
(12) repeating steps (8) to (10) to further improve the purity of the product, if necessary;
(13) performing final quality control testing as per US Pharmacopeia (USP); and
(14) yielding a highly purified solution or suspension with at least 99.5% twisted square antiprismatic (TSA′) structure.

18. A method for making a lanthanide ion dodecane tetraacetic acid (DOTA) complex contrast agent comprising steps of:
(1) adding an amount of 1,4,7,10-tetraazacyclo-dodecane-N, N′, N″, N′″-tetraacetic acid (DOTA) to purified water in a vessel to form a solution or suspension;
(2) stirring and simultaneously heating the solution or suspension to a temperature range between at least about 92° C. to about 98° C.;
(3) adding a lanthanide series oxide incrementally controlled to the stirring solution or suspension;
wherein:
each subsequent addition of the lanthanide series oxide occurs after the solution or suspension turns to a substantially clear color;
(4) repeating step (3) until a substantially equimolar amount of the lanthanide series oxide is added relative or comparative to the amount of DOTA previously added in step (1) to form a lanthanide oxide DOTA solution or suspension;
(5) reacting the lanthanide oxide DOTA solution formed in step (4) at a temperature of about 92° C. to 98° C. for at least 32 hours to obtain a lanthanide oxide DOTA complex having a twisted square antiprismatic (TSA) structure
(6) cooling the lanthanide oxide DOTA solution temperature of about 35° C. to 45° C.; and
(7) adding free base meglumine to the cooled lanthanide oxide DOTA solution to form in at least 99.5% yield a lanthanide oxide DOTA complex having a twisted square antiprismatic (TSA′) structure;
wherein:
the twisted square antiprismatic (TSA′) structure is maintained in a pH in a range between about 7.2 to 7.4.

19. The method for making a lanthanide ion dodecane tetraacetic acid (DOTA) complex contrast agent according to claim 18, further comprising additional purification steps of:
(8) taking:
the at least 99.5% yield a lanthanide oxide DOTA complex having the twisted square antiprismatic (TSA′) structure, a product formed in the solution or a suspension of step (7); and
adding:
at least 0.9 to 1.1 equivalents of a strong acid cation exchange resin; and
de-ionized water;
to form a combined new mixture solution or suspension that is stirred in the flask for at least an additional 20 minutes to 2 hours at a temperature between about 15° C. to 40° C.;
wherein:
the strong acid cation exchange resin assists in removing additional formed products and other residual materials in the new mixture solution or suspension by: dechelating non-ideal cages (non twisted square antiprismatic (TSA′) structures; sequestering any released Gd³⁺ ions; and/or protonated DOTA;
wherein: for each Gd³⁺ molecule sequestered, three meglumine+molecules is released into the new mixture solution or suspension;
(9) filtering the combined new mixture solution or suspension of step (8) to separate the strong acid cation exchange resin from and isolate the further purified product: lanthanide oxide DOTA complex having a twisted square antiprismatic (TSA′) structure of step (7) in the solution or suspension;
(10) adjusting and fine-tuning the solution and suspension by:
a. measuring the meglumine concentration and pH
b. adjusting pH back to about 7.2-7.4 by adding free base meglumine;
(11) performing analytical tests to quantify percentage of the further purified product of step (10);
(12) repeating steps (8) to (10) to further improve purity of the product, if necessary;
(13) performing final quality control testing as per US Pharmacopeia (USP); and
(14) yielding a highly purified solution or suspension with at least 99.5% twisted square antiprismatic (TSA′) structure.
